(19) United States
(12) Patent Application Publication
(10) Pub. No.: US 2012/0064024 A1
(43) Pub. Date: Mar. 15, 2012

(54) Precipitated Polymers

(75) Inventors: Kim Son Nguyen, Hemsbach (DE); Ina Fast, Frankenthal (DE); Rolf Werner, Ludwigshafen (DE)

(73) Assignee: BASF SE, Ludwigshafen (DE)

(21) Appl. No.: 13/320,596

(22) PCT Filed: May 12, 2010
(86) PCT No.: PCT/EP2010/056512

§ 371 Date: Nov. 15, 2011

(30) Foreign Application Priority Data
May 15, 2009 (EP) 09160344.9

(51) Int. Cl. A61K 8/81; A61Q 5/06; A61Q 5/02; C08F 226/10

(52) U.S. Cl. 424/70.15; 526/264; 510/126

(57) ABSTRACT

The present invention relates to precipitation polymers which comprise, in copolymerized form, 80 to 99.9% by weight of at least one nonionic water-soluble monomer a) and 0.1 to 20% by weight of a monomer b) carrying amide or urea groups. The invention further relates to the use of these polymers as rheology modifiers for in particular aqueous compositions and as rheology-modifying setting agents in hair cosmetics. The invention further relates to aqueous compositions comprising these polymers.

The present invention relates to precipitation polymers which comprise, in copolymerized form, 80 to 99.9% by weight of at least one nonionic water-soluble monomer a) and 0.1 to 20% by weight of a monomer b) carrying amide or urea groups. The invention further relates to the use of these polymers as rheology modifiers for in particular aqueous compositions and as rheology-modifying setting agents in hair cosmetics. The invention further relates to aqueous compositions comprising these polymers.

An object of the present invention is to provide products, in particular cosmetic products, having a complex profile of properties using the smallest possible amount of the respective active ingredient and/or the smallest possible number of different active ingredients.

In the hair cosmetic sector, there is a need for polymers which both permit an adjustment of the rheological properties of the preparations and also have a setting effect when used on the hair.

It is a further object of the present invention to provide polymers with the highest possible content of copolymerized N-vinylpyrrolidone, good setting properties when used in the hair and low stickiness which, moreover, can be advantageously prepared and further processed.

Polymers are often subjected to increased mechanical stress during their preparation and/or subsequent processing. For numerous polymers which are used in the aforementioned fields of application, such a stress often leads to very small particle sizes, which then bring about undesired dust formation.

EP-A 328725 and EP-A 814101 describe the preparation of acrylic-acid-based thickeners by precipitation polymerization.

U.S. Pat. No. 5,130,388 describes the preparation of acrylic acid-acrylamide copolymers by precipitation polymerization.

WO 2006/114404 describes a method of preparing precipitation polymers by spray polymerization of a monomer solution comprising at least one ethylenically unsaturated monomer a), at least one solvent b), optionally at least one crosslinker c) and optionally at least one initiator d), where monomer a) is soluble in the solvent b) and the polymer obtained by polymerization of a) is not soluble in the solvent b).

WO 2007/010035 describes the use of ampholytic copolymers as thickeners for cosmetic preparations, where the copolymers are prepared by precipitation polymerization.

WO 2003/092640 describes copolymers based on vinylpyrrolidone and methacrylamide which are obtained by solution polymerization.

WO 2005/123014 describes copolymers based on vinylpyrrolidone, vinylimidazole and methacrylamide which are obtained by free-radical solution polymerization.

U.S. Pat. No. 5,015,708 describes a method of preparing a terpolymer of (i) a vinyllactam, (ii) an acid-group-containing monomer and (iii) a hydrophobic monomer, which may, inter alia, be an ethylenically unsaturated silicone compound, by precipitation polymerization, and the preparation of powders from these polymers.

WO 04/058837 describes ampholytic copolymers obtainable by radical copolymerization of

a) at least one ethylenically unsaturated compound with at least one anionogenic and/or anionic group,
b) at least one ethylenically unsaturated compound with at least one cationogenic and/or cationic group,
c) at least one unsaturated amide-group-containing compound, and optionally further comonomers.

EP 1000610 A1 (BASF) describes solution polymers consisting of a) 0.05 to 90% by weight of monomers of the formula

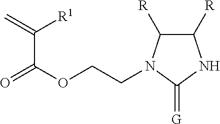

in which R¹ is H or CH₃, G is O or NH and R is H or identical or different organic radicals, which may also be bonded to one another, and b) 99.95 to 10% by weight of vinylcaprolactam and/or vinylpyrrolidone.

The object of the present invention was to provide novel polymers which are suitable for modifying the rheological properties of cosmetic compositions and as film formers and at the same time have a setting effect when used on hair. In particular, it should be possible to prepare and process these polymers essentially without dust formation.

Gel-like preparations for cosmetics should combine as many of the following properties as possible:
the resulting gels should be as clear as possible,
the resulting gels should be able to be distributed easily in the hair and give it good hold, which can be achieved particularly readily by gels with thixotropic properties,
the resulting gels should themselves have film-forming properties and thus contribute to setting of the hair,
the resulting gels should have conditioning, properties and improve the sensory properties of the hair, e.g. give it suppleness and shine and, after drying, not be sticky or only be slightly sticky,
the hair treated with the resulting gels should have good wet combability (thus the freshly treated hair can be readily shaped using a comb in order to form the desired hairstyle),
the polymers used for preparing the gels should make it possible for gels to be formulated in, as far as possible, all cosmetically acceptable pH ranges, specifically in the pH range from about 5 to 9,
the polymers used for the preparation of gels should permit the formulation of gels whose properties are pH-switchable,
the polymers used for the preparation of the gels should be able to be formulated together with standard commercial thickeners.

Surprisingly, it has been found that these objects are achieved by polymers obtainable by precipitation polymerization which comprise, in copolymerized form,
a) 80 to 99.9% by weight of at least one nonionic water-soluble monomer of the formula I

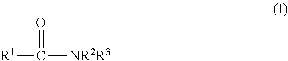

where
one of the radicals R¹ to R³ is a group of the formula CH₂═CR⁴— where R⁴═H or C₁-C₄-alkyl and the other radicals R¹ to R³, independently of one another, are H, alkyl, cycloalkyl, heterocycloalkyl, aryl or hetaryl,
where R¹ and R², together with the amide group to which they are bonded, may also be a lactam having 5 to 8 ring atoms,
where R² and R³, together with the nitrogen atom to which they are bonded, may also be a five- to seven-membered heterocycle,
with the proviso that the sum of the carbon atoms of the radicals R¹, R² and R³ is at most 8
and
b) 0.1 to 20% by weight of a monomer selected from
   b1) methacrylamide,
   b2) radically polymerizable compounds which comprise a structural element of the formula b2)

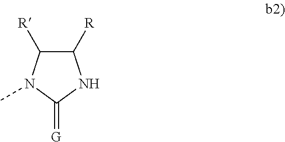

in which R and R′, independently of one another, are hydrogen, alkyl, cycloalkyl, heterocycloalkyl, aryl or hetaryl, preferably C₁-C₄-alkyl, where R and R′ may also be covalently bonded, and G is O or NH,
b3) polyhydroxy compounds carrying at least one allylamino group and
b4) mixtures thereof, where the total amount of all of the copolymerized monomers is 100% by weight.

Hereinbelow, the polymers according to the invention which are obtainable by precipitation polymerization are also referred to as “precipitation polymers”.

Without being limited to the description of the formula (I), the expression alkyl within the context of the present invention comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include in particular methyl, ethyl, propyl, isopropyl, n-butyl, 2-butyl, sec-butyl, tert-butyl, n-pentyl, 1-methylbutyl, 2-methylbutyl, 3-methylbutyl, 1,2-dimethyl-propyl, 1,1-dimethylpropyl, 2,2-dimethylpropyl, ethylpropyl, n-hexyl, 1-methylpentyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-tri-methylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl.

Without being limited to the description of the formula (I), the expression C₈-C₃₀-alkyl groups or C₈-C₃₀-alkenyl groups within the context of this invention generally comprises straight-chain and branched alkyl groups and alkenyl groups, respectively. These are preferably predominantly linear alkyl radicals, as also occur in natural or synthetic fatty acids and fatty alcohols and oxo alcohols, which may optionally be additionally mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), arachinyl(ene), behenyl(ene), lignocerinyl(ene), melissinyl(ene). Preference is also given to branched C₈-C₃₀-alkyl radicals, such as, for example, 2-ethylhexyl.

Without being limited to the description of the formula (I), the expression cycloalkyl within the context of this invention preferably comprises C₅-C₈-cycloalkyl, such as cyclopentyl, cyclohexyl, cycloheptyl or cyclooctyl.

Without being limited to the description of the formula (I), the expression aryl within the context of this invention generally comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

Within the context of the present invention, water-soluble monomers or water-soluble polymers are understood as meaning those monomers or polymers which dissolve in water at 20° C. to at least 1 gram per liter (g/l), preferably to at least 10 g/l, in such a way as to produce a solution that is clear to the human eye.

Within the context of this invention, water-dispersible monomers or water-dispersible polymers are generally understood as meaning those monomers or polymers which disintegrate into water-dispersible particles under the application of shear forces, for example by stirring. Hydrophilic monomers are preferably water-soluble or at least water-dispersible. The precipitation polymers according to the invention are generally water-soluble or water-dispersible; they are preferably water-soluble.

### Monomer a)

Preferably, the nonionic water-soluble monomers a) of the formula (I) have at most 7 further carbon atoms in addition to the carbonyl carbon atom of the amide group.

Monomer a) is preferably selected from primary amides of α,β-ethylenically unsaturated monocarboxylic acids, N-vinylamides of saturated monocarboxylic acids, N-vinyllactams, N-alkyl- and N,N-dialkylamides of α,β-ethylenically unsaturated monocarboxylic acids and mixtures thereof.

Preferred monomers a) are N-vinyllactams and derivatives thereof which can, for example, have one or more C₁-C₆-alkyl substituents, such as methyl, ethyl, n-propyl, isopropyl, n-butyl, sec-butyl, tert-butyl. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam.

Particular preference is given to using N-vinylpyrrolidone and/or N-vinylcaprolactam as monomer a).

The precipitation polymers according to the invention preferably comprise at least 92% by weight, further preferably at least 94% by weight and preferably at most 98, further preferably at most 96% by weight, of monomer a) in copolymerized form.

### Monomer b)

According to the invention, monomer b) is selected from

b1) methacrylamide,
b2) radically polymerizable compounds which comprise a structural element of the formula b2)

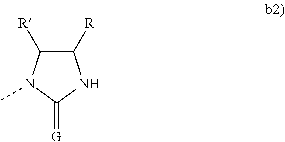

in which R and R′, independently of one another, are hydrogen, alkyl, cycloalkyl, heterocycloalkyl, aryl or hetaryl, where R and R′ may also be covalently bonded, and G is O or NH,
b3) polyhydroxy compounds carrying at least one allylamino group and
b4) mixtures thereof.

One embodiment of the invention is precipitation polymers according to the invention which comprise, as monomer b), b1) methacrylamide in copolymerized form.

A further embodiment of the invention is precipitation polymers according to the invention which comprise, in copolymerized form, as monomer b), b2) at least one radically polymerizable compound which comprises a structural element of the formula b2)

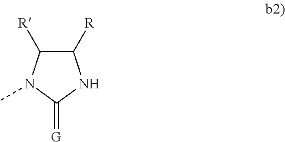

in which R and R′, independently of one another, are hydrogen, alkyl, cycloalkyl, heterocycloalkyl, aryl or hetaryl, where R and R′ may also be covalently bonded, and G is O or NH.
Suitable monomers b) are described in EP 1000610 A1 (BASF), in particular in paragraph [0011], to which reference is hereby made.
A particularly preferred monomer b2) is the compound of the following formula b2a)

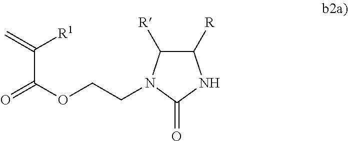

in which R, R′ and H are as defined above and R¹ is hydrogen or methyl. In one preferred embodiment of the invention, monomer b2) is 2-ethyl-(2-oxo-imidazolidin-1-yl)methacrylate, thus R and R′ are hydrogen and R¹ is methyl.

Within the context of this invention, 2-ethyl-(2-oxoimidazolidin-1-yl)methacrylate is also referred to as “ureido methacrylate” or in short “UMA”.

A preferred embodiment of the invention is precipitation polymers which comprise, in copolymerized form, from 0.1 to 5% by weight, preferably from 0.5 to 3% by weight, of ureido methacrylate.

Ureido methacrylate is preferably used as a mixture with methyl methacrylate, where such mixtures preferably comprise in the range from 20 to 60% by weight, preferably from 25 to 50% by weight, of UMA. Such mixtures are commercially available, for example as Norsocryl® (Arkema), Plex®6844-O (Röhm) or 25% UMA in methyl methacrylate MMA (BASF).

A further embodiment of the invention is precipitation polymers according to the invention which comprise, in copolymerized form, as monomer b3), a polyhydroxy compound carrying at least one allylamino group.

Within the context of the present invention, an allylamino group is preferably understood as meaning the structural unit —NR⁵—CH═CH₂, where R⁵ is hydrogen or C₁-C₄-alkyl, particularly preferably hydrogen.

Preferred aforementioned polyhydroxy compounds are compounds comprising at least three OH groups from the group consisting of polyglycerol, sugar carboxylic acids, alkylglucosides, mono- and oligosaccharides, which comprise up to four mono-saccharide units, sugar alcohols and oxidation products of said oligosaccharides, aminosorbitol, aminodisorbitol, glucosamine, N-acetylglucosamine, triethanolamine and trishydroxyethylmelamine, where these compounds in each case have at least one allylamino group.

Preferred polyglycerols are those which comprise 3 to 10 glycerol units. Such compounds are formed, for example, during the condensation of glycerol in the presence of alkali or acid. Within the context of the present invention, polyglycerol should also be understood as meaning the oligomers, such as triglycerol, tetraglycerol, pentaglycerol and hexaglycerol, and also polymers which have up to 10 glycerol units in the molecule, all of these compounds each having at least one allylamino group.

Suitable sugar carboxylic acids are preferably the oxidation products of sugars having 4 to 7 carbon atoms, e.g. gluconic acid, glucoheptonic acid, glucaric acid, galactaric acid, glucuronic acid or mannonic acid, and also the corresponding lactones, e.g. gluconolactone and glucoheptonolactone, these compounds each having at least one allylamino group.

Further suitable polyhydroxy compounds are in each case alkylglucosides and alkylpolyglucosides, alkylmaltosides and alkylmaltotriosides carrying at least one alkylamino group. The alkyl group may be C₁ to C₅, preferably a C₁- to C₄-alkyl group, for example methyl, ethyl, n-propyl, isopropyl, n-butyl or isobutyl. Moreover, the alkyl group may be substituted, e.g. carry a hydroxyl group. Suitable compounds of this type are, for example, hydroxyethylglucoside and hydroxypropylglucoside and the corresponding polyglucosides. The polyglucosides comprise on average 1.1 to 10, preferably 1.3 to 3 glucoside units.

Suitable oligosaccharides carrying at least one allylamino group and which comprise up to 4 monosaccharide units are, for example, maltose, maltotriose, maltotetraose, sucrose, lactose, leucrose, isomaltulose, chitobiose, chitotriose, chitotetraose carrying at least one allylamino group, and the derivatives obtainable therefrom by cleaving off the acetyl groups. Suitable monosaccharide units of the oligosaccharides are all customary monosaccharides, in particular units derived from glucose, galactose, fructose and mannose.

Sugar alcohols of oligosaccharides which comprise up to 4 monosaccharide units are obtainable from the aforementioned oligosaccharides through reduction. The oxidation products of said oligosaccharides include, for example, sucrose tricarboxylic acid and lactobionic acid.

Further suitable polyhydroxy compounds carrying at least one allylamino group are, moreover, monosaccharides carrying at least one allylamino group, such as, for example, glucose, galactose, mannose, and fructose, which each have at least one allylamino group.

Further suitable polyhydroxy compounds carrying at least one allylamino group are aminosorbitol, aminodisorbitol, glucosamine, N-acetylglucosamine, triethanolamine and trishydroxyethylmelamine, which each carry at least one allylamino group.

Preferred suitable polyhydroxy compounds carrying at least one allylamino group are polyglycerols having 3 to 10 glycerol units, gluconic acid, glucoheptonic acid, maltose and hydroxyethylglucoside, which each carry at least one allylamino group.

Preferred polyhydroxy compounds carrying at least one allylamino'group as monomers b) are the allylamides of sugar carboxylic acids.

Particularly preferred polyhydroxy compounds carrying at least one allylamino group as monomers b) are allylamides of sugar carboxylic acids, where the sugar carboxylic acid is selected from the group consisting of the oxidation products of sugars having 4 to 7 carbon atoms. Such sugar carboxylic acids are, for example, gluconic acid, glucoheptonic acid, glucaric acid, galactaric acid, glucuronic acid or mannonic acid.

The particularly preferred polyhydroxy compound carrying allylamino groups as monomer b) is allyl-D-gluconamide of the formula II

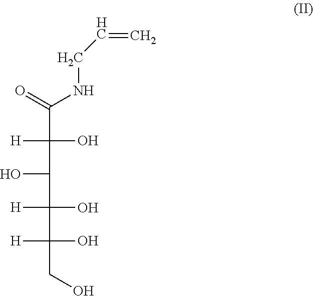

A further embodiment of the invention is thus precipitation polymers according to the invention which comprise copolymerized allyl-D-gluconamide as monomer b).

### Further Monomers

The precipitation polymers according to the invention can comprise, in copolymerized form, from 0% by weight to 19.9% by weight, preferably from 0.5 to 15% by weight, of further monomers c) which are different from a) and b).

It is critical that the total amount of all of the monomers copolymerized into the precipitation polymers according to the invention is 100% by weight.

c1) Anionic or Anionogenic Monomers

Preferred further monomers c) are compounds c1) with a radically polymerizable, α,β-ethylenically unsaturated double bond and at least one anionogenic and/or anionic group per molecule. Monomers comprising acid groups are referred to in the uncharged state as anionogenic, and in the charged state as anionic.

One embodiment of the invention is thus precipitation polymers according to the invention which comprise, in copolymerized form, at least 0% by weight to 19.9% by weight of a monomer c1), the sum of all of the copolymerized monomers being 100% by weight.

Preferred further monomers c) are compounds with a radically polymerizable, α,β-ethylenically unsaturated double bond and at least one anionogenic and/or anionic group per molecule. Monomers comprising acid groups are referred to in the uncharged state as anionogenic, and in the charged state as anionic.

Preferred compounds c1) are compounds which are selected from monoethylenically unsaturated carboxylic acids, sulfonic acids, phosphonic acids and mixtures thereof. These include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The compounds c1) further include the half-esters of monoethylenically unsaturated dicarboxylic acids having 4 to 10, preferably 4 to 6, carbon atoms, e.g. of maleic acid, such as monomethyl maleate. The compounds c1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropane-sulfonic acid, vinylphosphonic acid and allylphosphonic acid. The compounds c1) also include the salts of the aforementioned acids, in particular the sodium, potassium and ammonium salts and also the salts with amines. These monomers c1) can be used as such or as mixtures.

Preferred monomers c1) are selected from the group consisting of acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid, 2-acrylamido-2-methylpropanesulfonic acid, vinylphosphonic acid and mixtures thereof.

Monomer c1) is particularly preferably selected from the group consisting of acrylic acid, methacrylic acid and mixtures thereof.

In one embodiment of the invention, the polymers according to the invention comprise acrylic acid in copolymerized form. In one preferred embodiment of the invention, the polymers according to the invention comprise from 1 to 15% by weight, further preferably from 2 to 10% by weight, of acrylic acid in copolymerized form.

c2) Vinylimidazoles

One embodiment of the invention is precipitation polymers according to the invention which comprise, in copolymerized form, from 0% by weight to 19.9% by weight, preferably from 0.5 to 15% by weight, of at least one monomer c2), the sum of all of the copolymerized monomers being 100% by weight.

Preferred monomers c2) are monomers of the general formula IIa

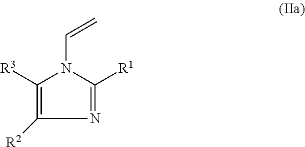

where R¹ to R³, independently of one another, are hydrogen, C₁-C₄-alkyl or phenyl.

Examples of monomers c2) can be found in table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Particularly preferred monomers c2) are 1-vinylimidazole (N-vinylimidazole) and mixtures which comprise N-vinylimidazole.

One embodiment of the invention is polymers according to the invention which comprise, in copolymerized form, from 2 to 15, preferably from 5 to 10%, by weight of 1-vinylimidazole, the sum of all of the copolymerized monomers being 100% by weight.

c3) Esters of (Meth)Acrylic Acid C₁-C₂₄-Alkyl-Substituted on the Nitrogen

Preferred further monomers c) are c3) esters of (meth)acrylic acid with amino alcohols mono- or di-C₁-C₂₄-alkyl-substituted on the nitrogen (monomers c3). Particular preference is given to the monomers c3) selected from the group consisting of N-methylaminoethyl (meth)acrylate, N-ethylaminoethyl (meth)acrylate, N-(n-propyl)-aminoethyl (meth)acrylate, N-(n-butyl)aminoethyl (meth)acrylate, N-(tert-butyl)-aminoethyl (meth)acrylate, N,N-dimethylaminomethyl (meth)acrylate, N,N-dimethyl-aminoethyl (meth)acrylate, N,N-diethylaminoethyl (meth)acrylate, N,N-dimethylamino-propyl (meth)acrylate, N,N-diethylaminopropyl (meth)acrylate and N,N-dimethylamino-cyclohexyl (meth)acrylate.

Particularly preferred as monomer c3) is N,N-dimethylaminoethyl methacrylate (DMAEMA).

One embodiment of the invention is thus precipitation polymers according to the invention which comprise at least one monomer c3) in copolymerized form. A further embodiment of the invention is polymers according to the invention which comprise, in copolymerized form, from 2 to 15% by weight, preferably from 5 to 10% by weight, of DMAEMA, the sum of all of the copolymerized monomers being 100% by weight.

c4) Amides of (Meth)Acrylic Acid C₁-C₂₄-Alkyl-Substituted on the Nitrogen

Preferred further monomers c) are c4) amides of (meth)acrylic acid with diamines mono- or di-C₁-C₂₄-alkyl-substituted on the nitrogen (c4). Particular preference is given to the monomers c4) selected from the group consisting of N-[2-(dimethylamino)ethyl]-acrylamide, N-[2-(dimethylamino)ethyl]methacrylamide, N-[3-(dimethylamino)propyl]-acrylamide, N-[3-(dimethylamino)propyl]methacrylamide, N-[4-(dimethylamino)butyl]-acrylamide, N-[4-(dimethylamino)butyl]methacrylamide, N-[2-(diethylamino)ethyl]-acrylamide, N-[4-(dimethylamino)cyclohexyl]acrylamide, N-[4-(dimethylamino)cyclo-hexyl]methacrylamide, N-[8-(dimethylamino)octyl]methacrylamide, N-[12-(dimethyl-amino)dodecyl]methacrylamide, N-[3-(diethylamino)propyl]methacrylamide and N-[3-(diethylamino)propyl]acrylamide.

A particularly preferred monomer c4) is N-[3-(dimethylamino)propyl]methacrylamide (DMAPMAM).

One embodiment of the invention is thus precipitation polymers according to the invention which comprise at least one monomer c4) in copolymerized form.

A further embodiment of the invention is polymers according to the invention which comprise, in copolymerized form, from 2 to 15% by weight, preferably from 5 to 10% by weight, of DMAPMAM, the sum of all of the copolymerized monomers being 100% by weight.

c5) N,N-Diallylamines and N,N-diallyl-N-alkylamines

Preferred further monomers c) are c5) N,N-diallylamines and N,N-diallyl-N-alkylamines and acid addition salts and quaternization products thereof (monomers c5)). Alkyl here is preferably C₁-C₂₄-alkyl. Preferred monomers c5) are N,N-diallyl-N-methylamine and N,N-diallyl-N,N-dimethylammonium compounds, such as, for example, the chlorides and bromides. Preferred monomers c5) include in particular N,N-diallyl-N-methylamine and its methylated derivative N,N-diallyl-N,N-dimethylammonium chloride (DADMAC). One embodiment of the invention is thus precipitation polymers according to the invention which comprise at least one monomer c5) in copolymerized form, the sum of all of the copolymerized monomers being 100% by weight.

A further embodiment of the invention is polymers according to the invention which comprise, in copolymerized form, from 2 to 15, preferably from 5 to 10%, by weight of DADMAC, the sum of all of the copolymerized monomers being 100% by weight.

The monomers of groups c2) to c5), such as, for example, VI, DMAPMAM, DMAEMA or diallyl-N-methylamine are usually referred to in the uncharged state as cationogenic, and in the charged state as cationic. Within the context of this invention, the term “cationic” is sometimes also used instead of the term “cationogenic”.

c6) Open-Chain N-Vinylamide Compounds

Preferred further monomers c) are, for example, open-chain N-vinylamide compounds c6), such as, for example, N-vinylformamide, N-vinyl-N-methylformamide, N-vinyl-acetamide, N-vinyl-N-methylacetamide, N-vinyl-N-ethylacetamide, N-vinyl-propionamide, vinyl-N-methylpropionamide, N-vinylbutyramide and mixtures thereof. One embodiment of the invention is thus precipitation polymers according to the invention which comprise at least one monomer c6) in copolymerized form, the sum of all of the copolymerized monomers being 100% by weight.

c7) Esters and Amides of α,β-Ethylenically Unsaturated Mono- and Dicarboxylic Acids

Preferred monomers c) are also c7) esters of α,β-ethylenically unsaturated mono- and dicarboxylic acids with C₁-C₃₀-alkanols, preferably'C₁-C₂₂-alkanols. Preferred monomers c7) are also amides of α,β-ethylenically unsaturated mono- and dicarboxylic acids with mono- and dialkylamines which have 1 to 30 carbon atoms, preferably 1 to 22 carbon atoms, per alkyl radical. The monomers c7) are preferably compounds of the general formula III

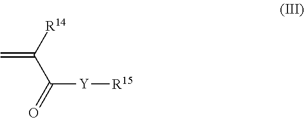

in which
R¹⁴ is hydrogen or C₁- to C₈-alkyl,
R¹⁵ is a straight-chain or branched C₁- to C₃₀-alkyl radical, and
Y is O or NR¹⁸, where R¹⁸ is hydrogen, C₁- to C₈-alkyl or C₅- to C₈-cycloalkyl.

Preferably, in formula III, R¹⁴ is hydrogen, methyl or ethyl.

Preferably, Y is O or NH.

Suitable radicals R¹⁵ are the aforementioned C₁-C₃₀-alkyl radicals. In particular, R¹⁵ is methyl, ethyl, n-propyl, isopropyl, n-butyl, 2-butyl, sec-butyl, tert-butyl, n-pentyl, n-hexyl, n-heptyl, n-octyl, ethylhexyl, 1,1,3,3-tetramethylbutyl, undecyl, lauryl, tridecyl, myristyl, pentadecyl, palmityl, margarinyl, stearyl, palmitoleinyl, oleyl or linolyl.

In particular, monomer c7) is selected from methyl (meth)acrylate, methyl ethacrylate, ethyl (meth)acrylate, ethyl ethacrylate, tert-butyl (meth)acrylate, tert-butyl ethacrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate, tert-butyl(meth)acrylamide, N,N-dimethyl(meth)acrylamide, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleyl(meth)acrylamide, oleyl(meth)acrylamide, linolyl(meth)acrylamide, linolenyl(meth)acrylamide, stearyl(meth)acrylamide, lauryl(meth)acrylamide and mixtures thereof.

A further embodiment of the invention is polymers according to the invention which comprise, in copolymerized form, from 1 to 15% by weight of methyl methacrylate, the sum of all of the copolymerized monomers being 100% by weight.

Further suitable monomers c7) are hydroxy-substituted (meth)acrylic acid esters and hydroxy-substituted methacrylamides, i.e. compounds of the formula III, where R¹⁵ is a straight-chain or branched hydroxy-substituted C₁- to C₃₀-alkyl radical.

Suitable hydroxy-substituted (meth)acrylic acid esters c7) include, for example, 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethyl-hexyl methacrylate.

Suitable hydroxy-substituted (meth)acrylamides c7) include, for example, 2-hydroxy-ethylacrylamide, 2-hydroxyethylmethacrylamide, 2-hydroxyethylethacrylamide, 2-hydroxypropylacrylamide, 2-hydroxypropylmethacrylamide, 3-hydroxypropyl-acrylamide, 3-hydroxypropylmethacrylamide, 3-hydroxybutylacrylamide, 3-hydroxy-butylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethyl-hexylacrylamide and 3-hydroxy-2-ethylhexylmethacrylamide.

One embodiment of the invention is thus precipitation polymers according to the invention which comprise at least one monomer c7) in copolymerized form, the sum of all of the copolymerized monomers being 100% by weight.

Compounds c8)

One embodiment of the invention is precipitation polymers according to the invention which comprise at least one monomer c8) in copolymerized form. Compounds c8) are selected from compounds of the general formulae c8-1) and c8-2)

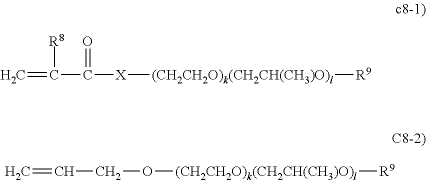

in which
the order of the alkylene oxide units is arbitrary,
k and l, independently of one another, are an integer from 0 to 1000, where the sum of k and l is at least 5,
R⁸ is hydrogen or C₁-C₄-alkyl, preferably methyl,
R⁹ is C₈-C₃₀-alkyl or C₈-C₃₀-alkenyl, and
X is O or a group of the formula NR¹⁸, in which R¹⁹ is H, alkyl, alkenyl, cycloalkyl, heterocycloalkyl, aryl or hetaryl.

In the formulae c8-1) and c8-2), k is preferably an integer from 1 to 500, in particular 3 to 250. Preferably, I is an integer from 0 to 100.

Preferably, R⁸ in of the formula c8-1) is hydrogen, methyl, ethyl, n-propyl, isopropyl, n-butyl, sec-butyl, tert-butyl, n-pentyl or n-hexyl, in particular hydrogen, methyl or ethyl.

Preferably, R⁹ in the formulae c8-1) and c8-2) is n-octyl, 1,1,3,3-tetramethylbutyl, ethyl-hexyl, n-nonyl, n-decyl, n-undecyl, tridecyl, myristyl, pentadecyl, palmityl, heptadecyl, octadecyl, nonadecyl, arrachinyl, behenyl, lignocerenyl, cerotinyl, melissinyl, palmitoleinyl, oleyl, linolyl, linolenyl, stearyl, lauryl.

Preferably, X in the formula c8-1) is O or NH.

Suitable polyether acrylates of the formula c8-1) are, for example, the polycondensation products of the aforementioned α,β-ethylenically unsaturated mono- and/or dicarboxylic acids and their acid chlorides, amides and anhydrides with polyetherols. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The polyether acrylates of the formula c8-1) can be used alone or in mixtures for the preparation of the precipitation polymers according to the invention.

Preferred polyether acrylates of the formula c8-1) are esters of methacrylic acid with ethoxylated C₁₆-C₂₂-fatty alcohol mixtures. Particular preference is given to esters of methacrylic acid with ethoxylated C₁₆-C₁₈-fatty alcohol mixtures, the degree of ethoxylation being approximately 25.

Preferred polyether acrylates are compounds of the formula c8-1), where R⁸ is methyl, X is oxygen, R⁹ is C₁₆-C₁₈-alkyl, k=25 and l=zero: C₁₆-C₁₈-alkyl PEG₁₁₀₀ methacrylate.

Suitable allyl alcohol alkoxylates c8-2) are, for example, the etherification products of allyl chloride with corresponding polyetherols. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The allyl alcohol alkoxylates c8-2) can be used alone or in mixtures for the preparation of the polymers according to the invention.

One embodiment of the invention is polymers according to the invention which comprise, in copolymerized form, from 0.1 to 10% by weight, preferably from 0.5 to 3% by weight, of a compound c8), preferably of the formula c8-1), the sum of all of the copolymerized monomers being 100% by weight.

c9) Urethane (Meth)Acrylates

Suitable monomers c) are also c9) urethane (meth)acrylates, as described, for example, in DE-A 198 38 852, p. 3, I. 45 to p. 9, I. 20, to which reference is hereby made in its entirety. However, the Si component referred to therein as d) is not necessarily a constituent of those urethane (meth)acrylates which can be used as monomer c9) within the context of the present invention.

A further embodiment of the invention is thus precipitation polymers according to the invention which comprise, in copolymerized form, at least one monomer c9), the sum of all of the copolymerized monomers being 100% by weight.

Suitable further monomers c) are ethylene, propylene, isobutylene, butadiene, styrene, α-methylstyrene, (meth)acrylonitrile, vinyl chloride, vinylidene chloride, vinyl fluoride, vinylidene fluoride and mixtures thereof.

The aforementioned additional monomers c) can in each case be used individually or in the form of any desired mixtures.

### Precipitation Polymerization

The present invention further provides a method of preparing the polymers according to the invention, wherein the method comprises a precipitation polymerization.

In the precipitation polymerization, the monomers used are soluble in the reaction medium which comprises the monomers and the solvent, but the resulting polymer is not. The resulting polymer is insoluble under the selected polymerization conditions and precipitates out. Here, it is possible to obtain copolymers with higher molecular weights than in accordance with other polymerization methods, e.g. by solution polymerization. Such copolymers with higher molecular weights are particularly advantageously suitable as rheology modifiers, in particular as thickeners.

The precipitation polymerization preferably takes place in a solvent in which each of the monomers used dissolves at 20° C. and 1 bar in an amount of at least 10% by weight to give a solution that is clear to the human eye.

Within the context of the present invention, a polymer is insoluble in a liquid phase if less than 1 gram, preferably less than 0.1 gram, of the polymer dissolves in one liter of the liquid phase to give a solution that is clear to the human eye.

The precipitation polymerization preferably takes place in a largely anhydrous, aprotic solvent or solvent mixture. A largely anhydrous, aprotic solvent or solvent mixture is understood as meaning a solvent or solvent mixture with a water content of at most 5% by weight.

Such aprotic solvents or solvent mixtures are preferably esters such as ethyl acetate and/or n-butyl acetate and/or hydrocarbons such as cyclohexane or n-heptane.

In one embodiment of the invention, the precipitation polymerization takes place in a solvent consisting of or comprising ethyl acetate.

In one embodiment of the invention, the precipitation polymerization takes place in a solvent consisting of or comprising n-butyl acetate.

In a further embodiment of the invention, the precipitation polymerization takes place in a solvent mixture of at least one ester and at least one hydrocarbon. A preferred solvent mixture is a mixture comprising or consisting of 80-90% by weight of ethyl acetate and 10-20% by weight of cyclohexane.

Preferably, the precipitation polymerization takes place at a temperature in the range from 70 to 140° C., preferably 75 to 100° C., in particular from 80 to 95° C. The resulting polymer particles precipitate out of the reaction solution and can be isolated by customary methods, such as filtration by means of subatmospheric pressure. For the precipitation polymerization it is possible to use surface-active, polymeric compounds, preferably based on polysiloxane. In the precipitation polymerization, polymers are generally obtained which have higher molecular weights than in the case of solution polymerization.

The polymerization usually takes place under atmospheric pressure, although it can also proceed under reduced or increased pressure. A suitable pressure range is between 1 and 5 bar.

### Initiators

Initiators which can be used for the radical polymerization are the peroxo and/or azo compounds customary for this, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide; tert-butyl peroctoate, azobisisobutyronitrile, azobis(2-amidonopropane)dihydrochloride, 2,2′-azobis(2-methylbutyronitrile) or 2,2′-azobis(2,4-dimethylvaleronitrile) (Wako® V65). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu(I).

In one embodiment of the invention, for preparing the polymers according to the invention, at least two radical initiators are used whose decomposition temperatures and/or their half-lives at a certain polymerization temperature are different from one another. Here, copolymers with particularly low residual monomer contents can be attained. This is the case particularly if the initiator that decomposes at the higher temperature is added before the polymer has finished precipitating, preferably before the polymer has started to precipitate.

Preferably, for the copolymerization, at least two initiators are used whose decomposition temperatures are different from one another by at least 10° C. Within the context of the invention, the decomposition temperature is defined as the temperature at which 50% of the molecules decompose into free radicals within 2.5 hours.

Preferably, the copolymerization takes place during this procedure until the copolymer has finished precipitating at a temperature greater than or equal to the lower decomposition temperature and lower than the higher decomposition temperature, and, after the precipitation, a further reaction takes place at a temperature greater than or equal to the higher decomposition temperature.

Preferably, the method according to the invention comprises a first polymerization phase at a first polymerization temperature and a second polymerization phase at a second polymerization temperature above the first polymerization temperature, where, for the polymerization, at least two initiators are used whose half-lives at the first polymerization temperature differ in such a way that at least one of these initiators decomposes into radicals during the first polymerization phase and at least one of these initiators essentially does not decompose into radicals during the first polymerization phase and does decompose into radicals during the second polymerization phase. Preferably, in the case of this procedure, the second polymerization phase starts substantially after precipitation of the copolymer.

“Substantially” after precipitation of the copolymer is understood as meaning that the copolymer is present in precipitated form preferably to at least 80% by weight, preferably to at least 90% by weight, in particular at least 95% by weight, based on the total weight of the copolymer.

The half-life of an initiator can be determined by customary methods known to the person skilled in the art, as described, for example, in the publication “Initiators for high polymers”, Akzo Nobel, No. 10737. The half-life of the first polymerization initiator at the first polymerization temperature and of the second polymerization initiator at the second polymerization temperature is preferably in a range from about 1 minute to 3 hours, particularly preferably 5 minutes to 2.5 hours. If desired, shorter half-lives, e.g. from 1 second to 1 minute or half-lives longer than 3 hours can also be used provided it is ensured that the initiator(s) decomposing at the higher temperature substantially decomposes into radicals during the second polymerization phase.

In addition to the first and second polymerization phase, further polymerization phases can be used at polymerization temperatures different therefrom. Thus, for example, it is possible to carry out a first polymerization phase at a first polymerization temperature which is selected such that a controlled polymerization (i.e. e.g. with avoidance of an undesired temperature increase as a result of the heat of reaction, of an excessively high reaction rate, etc.) takes place. Then, for example, an afterpolymerization can follow at a temperature which is above the first and below the second polymerization temperature and which is selected such that the initiator(s) decomposing at the higher temperature substantially do not decompose into radicals. Following the conclusion of this afterpolymerization, to which, if desired, the initiator decomposing at the lower temperature and/or another initiator decomposing under the conditions of the afterpolymerization can again be added, the second polymerization phase can then follow.

Preferably, the initiator system used comprises at least two initiators whose decomposition temperatures differ from one another by at least 15° C.

The initiator decomposing at the lower temperature preferably has a decomposition temperature of from 50 to 100° C.

The initiator decomposing at the higher temperature preferably has a decomposition temperature of from 80 to 50° C.

Preferably, the initiator decomposing at the higher temperature is initially introduced at the start of the copolymerization or is added before or during precipitation of the copolymer.

Preferably, the initiator decomposing at the higher temperature is initially introduced at the start of the copolymerization or added before precipitation of the copolymer.

In the case of a preferred initiator combination, the initiator decomposing at the lower temperature is Trigonox® EHP (bis(2-ethylhexyl) peroxydicarbonate, CAS No. 16111-62-9) and the initiator decomposing at the higher temperature is selected from tert-butyl peroxypivalate (e.g. Luperox® 11 M75 from Atochem), tert-butyl peroctoate, lauroyl peroxide (LPO, CAS No. 105-74-8) or 2,5-dimethyl-2,5-bis(t-butylperoxy)hexane (Trigonox® 101).

A further preferred initiator combination comprises Trigonox® EHP or 2,2′-azobis(2.4-dimethylvaleronitrile) (Wako® V65) and tert-butyl peroctoate.

A further preferred initiator combination comprises lauroyl peroxide and tert-butyl peroctoate and 2,5-dimethyl-2,5-bis(t-butylperoxy)hexane (Trigonox® 101).

A further preferred initiator combination comprises tert-butyl peroxypivalate (Luperox 11 M75 and tert-butyl peroctoate or 2,5-dimethyl-2,5-bis(t-butylperoxy)hexane (Trigonox® 101).

A further preferred initiator combination comprises tert-butyl peroctoate and 2,5-dimethyl-2,5-bis(t-butylperoxy)hexane (Trigonox® 101).

### Crosslinker e)

The precipitation polymers according to the invention can, if desired, comprise, in copolymerized form, at least one crosslinker e), i.e. a compound with two or more than two ethylenically unsaturated, nonconjugated double bonds.

Preferably, crosslinkers are used in an amount of from 0.01 to 1% by weight, particularly preferably 0.1 to 0.5% by weight, based on the total weight of the monomers used for the polymerization.

Suitable crosslinkers are, for example, those specified in WO 2007/010035, p. 17, I. 20 to p. 19, I. 18, to which reference is hereby made in its entirety.

Very particularly preferred crosslinkers e) are ethylene glycol di(meth)acrylate, poly-ethylene glycol di(meth)acrylates, pentaerythritol triallyl ether, methylenebisacrylamide, N,N′-divinylethyleneurea, triallylamine and triallylmonoalkylammonium salts. A most preferred crosslinker e) is pentaerythritol triallyl ether.

Within the context of this invention, the totality of all of the substances which are already located in the reaction vessel prior to the onset of the polymerization is referred to as the initial charge. This initial charge preferably comprises at least one solvent. However, in addition to the solvent, the initial charge can also already comprise one or more monomers. The initial charge can also comprise further substances, such as initiator, crosslinker or regulator.

In one embodiment of the present invention, the initial charge comprises solvent, some of the monomers and some of the initiator.

In a further embodiment of the invention, the initial charge comprises in the range from 10 to 30% by weight, preferably in the range from 15 to 25% by weight, of the total amount of all of the monomers to be copolymerized.

In a further embodiment of the invention, the initial charge comprises in the range from 10 to 20% by weight, preferably in the range from 13 to 17% by weight, of the total amount of the initiator to be used.

In a further embodiment of the invention, the initial charge comprises solvent and some of the monomers, but no initiator.

In a further embodiment of the invention, the initial charge does comprise solvent but neither monomers nor initiator.

In one embodiment of the invention, the amount of substances in the reaction mixture that are different from solvents is in the range from 10 to 30% by weight, preferably in the range from 15 to 25% by weight, particularly preferably in the range from 18 to 22% by weight, in each case based on the total weight of the reaction mixture. The fraction of substances in the reaction mixture that are different from solvents is also referred to as solids content (in short “SC”).

To achieve the purest possible polymers with a low residual monomer content, the polymerization (main polymerization) can be followed by an afterpolymerization step. The afterpolymerization can take place in the presence of the same initiator system as the main polymerization or in the presence of a different initiator system to the main polymerization. Preferably, the afterpolymerization takes place at least at the same, preferably at a higher, temperature than the main polymerization. The temperature during the main polymerization and the afterpolymerization is preferably at most 100° C. (main reaction) and 130° C. (afterpolymerization).

The precipitated polymer is separated off from the reaction mixture after the polymerization and/or after the afterpolymerization step.

For this separation, any customary method can be used for separating off polymers during conventional precipitation polymerization.

Suitable methods for separating off the precipitation polymers from the other constituents of the reaction mixture are, for example, filtration, centrifugation, evaporation of the solvent or combinations of these methods.

To further purify the precipitation polymers, a washing of the polymers is preferably carried out. For this, preference is given to using the same or similar solvents as have already been used for the preceding precipitation polymerization.

Washing of the precipitation polymers is an operation known to the person skilled in the art.

In one preferred embodiment of the invention, the polymers are dried.

This drying can take place in various ways.

The drying methods known to the person skilled in the art, such as, for example, heating, storing under reduced pressure, storing under customary standard conditions and combinations of these methods are suitable for this.

Compared to solution polymers, the pulverulent precipitation polymers according to the invention have the advantage of better storability and easier transportability and generally exhibit a lower propensity for microbial attack.

The preferably obtained polymer dry powders can advantageously be converted into an aqueous polymer solution or polymer dispersion through dissolution or dispersion in water.

If the precipitation polymers according to the invention comprise acid groups, then these can be partially or completely neutralized with a base. Bases which can be used for the neutralization of the polymers are alkali metal bases, such as sodium hydroxide solution, potassium hydroxide solution, sodium carbonate, sodium hydrogencarbonate, potassium carbonate or potassium hydrogencarbonate, and alkaline earth metal bases, such as calcium hydroxide, calcium oxide, magnesium hydroxide or magnesium carbonate, and also amines. Suitable amines are, for example, C₁-C₆-alkylamines, preferably n-propylamine and n-butylamine, dialkylamines, preferably diethylpropylamine and dipropylmethylamine, trialkylamines, preferably triethylamine and triisopropylamine. Preference is given to amino alcohols, e.g. trialkanolamines, such as triethanolamine, alkyldialkanolamines, such as methyl- or ethyldiethanolamine and dialkylalkanolamines, such as dimethylethanolamine, and 2-amino-2-methyl-1-propanol. Particularly for use in hair treatment compositions, 2-amino-2-methyl-1-propanol (abbreviated to “AMP”), 2-amino-2-ethylpropane-1,3-diol, diethylaminopropylamine and triisopropanolamine have proven useful for the neutralization of these polymers comprising acid groups. Likewise suitable are the bases specified in WO 03/099253, p. 2, line 20 to p. 3, line 6, to which reference is hereby made. The neutralization of the acid groups can also be carried out with the help of mixtures of two or more bases, e.g. mixtures of sodium hydroxide solution and triisopropanolamine. Neutralization can take place partially or completely depending on the intended application.

If the precipitation polymers according to the invention comprise cationogenic groups, such as, for example, amino groups, then it is possible, from these amino groups, by means of protonation, e.g. with mono- or polyhydric carboxylic acids such as lactic acid or tartaric acid or with mineral acids such as phosphoric acid, sulfuric acid and hydrochloric acid, or by means of quaternization, e.g. with alkylating agents, such as C₁- to C₄-alkyl halides or sulfates, to produce cationic groups.

Examples of suitable alkylating agents are ethyl chloride, ethyl bromide, methyl chloride, methyl bromide, dimethyl sulfate and diethyl sulfate.

If the precipitation polymers according to the invention are to be both quaternized and neutralized, then preferably firstly the quaternization takes place and then the neutralization.

One embodiment of the invention is precipitation polymers according to the invention, wherein monomer a) is or comprises N-vinylpyrrolidone (within the context of this invention also referred to in short as “VP”).

One embodiment of the invention is precipitation polymers which comprise, in copolymerized form as at least one monomer a), N-vinylpyrrolidone (within the context of this invention also referred to in short as “VP”) and, as at least one monomer c1) (meth)acrylic acid.

One embodiment of the invention is precipitation polymers which comprise, in copolymerized form, as a monomer a); N-vinylpyrrolidone and, as a monomer c2) N-vinylimidazole (within the context of this invention also referred to in short as “VI”).

One embodiment of the invention is precipitation polymers which comprise, in copolymerized form, as a monomer a), N-vinylpyrrolidone and, as a monomer c3), DMAEMA.

One embodiment of the invention is precipitation polymers which comprise, in copolymerized form, as a monomer a), N-vinylpyrrolidone and, as a monomer c4), DMAPMAM.

One embodiment of the invention is precipitation polymers which comprise, in copolymerized form, as a monomer a), N-vinylpyrrolidone, as a first monomer c), (meth)acrylic acid and, as a second monomer c), one or more from VI, DMAPMAM and DMAEMA. Preference is given here to a weight ratio of anionic/anionogenic monomer to cationic/cationogenic monomer of less than or equal to 1:2 or greater than or equal to 2:1.

A further embodiment of the invention is precipitation polymers which comprise, in copolymerized form,

a) 90 to 99.5% by weight of N-vinylpyrrolidone,
b) 0.5 to 10% by weight of methacrylamide (MAM)
c) c1) 0 to 10% by weight of (meth)acrylic acid and
c2) 0 to 10% by weight of N-vinylimidazole, the total amount of all of the copolymerized monomers being 100% by weight.

A further embodiment of the invention is precipitation polymers which comprise, in copolymerized form,

a) 85 to 97% by weight of N-vinylpyrrolidone,
b) 0.5 to 5% by weight of methacrylamide,
c) c2) 1.5 to 10% by weight of N-vinylimidazole, the total amount of all of the copolymerized monomers being 100% by weight.

A further embodiment of the invention is precipitation polymers which comprise, in copolymerized form,

a) 85 to 97% by weight of N-vinylpyrrolidone,
b) 0.5 to 5% by weight of methacrylamide,
c) c1) 1.5 to 10% by weight of acrylic acid, the total amount of all of the copolymerized monomers being 100% by weight.

A further embodiment of the invention is precipitation polymers which comprise, in copolymerized form,

a) 80 to 95% by weight of N-vinylpyrrolidone,
b) 0.2 to 5% by weight of ureido methacrylate (UMA)
c) c1) 1.5 to 10% by weight of (meth)acrylic acid and
c8-1) 0 to 3% by weight of C₁₆-C₁₈-alkyl PEG₁₁₀₀ methacrylate, the total amount of all of the copolymerized monomers being 100% by weight.

A further embodiment of the invention is precipitation polymers which comprise, in copolymerized form,

a) 80 to 95% by weight of N-vinylpyrrolidone,
b) 0.2 to 5% by weight of ureido methacrylate (UMA)
c) c1) 1.5 to 10% by weight of N-vinylimidazole (VI)
c8-1) 0 to 3% by weight of C₁₆-C₁₈-alkyl PEG₁₁₀₀ MA and
   d) 0 to 10% by weight of methyl methacrylate (c7) and/or methacrylic acid (c1), the total amount of all of the copolymerized monomers being 100% by weight.

In a further embodiment of the invention, the aforementioned precipitation polymers have in the range from 0.01 to 1% by weight, preferably in the range from 0.1 to 0.5% by weight, of a crosslinker, preferably pentaerythritol triallyl ether (PETAE), in copolymerized form, the total amount of all of the copolymerized monomers being 100% by weight.

In a further embodiment of the invention, the aforementioned precipitation polymers have, as monomer b), b1) methacrylamide and b3) allylgluconamide in copolymerized form.

In a further embodiment of the invention, the aforementioned precipitation polymers have, as monomer b), b3) allylgiuconamide instead of b1) methacrylamide in copolymerized form.

In a further embodiment of the invention, the aforementioned precipitation polymers have, as monomer b), b2) ureido methacrylate in copolymerized form.

A further embodiment of the invention is nonionic precipitation polymers comprising 95-99% by weight of N-vinylpyrrolidone and 1-5% by weight of methacrylamide (MAM) in copolymerized form, the total amount of all of the copolymerized monomers being 100% by weight, such as, for example:

| Precipitation polymer | VP | MAM |
|---|---|---|
| N1 | 98 | 2 |
| N2 | 95 | 5 |

A further embodiment of the invention is cationic/cationogenic precipitation polymers comprising 88-92% by weight of N-vinylpyrrolidone (VP), 3-6% by weight of methacrylamide (MAM), 4-8% by weight of a cationic/cationogenic monomer c), preferably N-vinylimidazole (VI) and 0-2% by weight of a crosslinker, preferably pentaerythritol triallyl ether (PETAE), in copolymerized form, the total amount of all of the copolymerized monomers being 100% by weight, such as, for example:

| Precipitation polymer | VP | MAM | VI | PETAE |
|---|---|---|---|---|
| K1 | 90 | 3 | 7 | |
| K2 | 90 | 5 | 5 | |
| K3 | 90 | 3 | 6.8 | 0.2 |

A further embodiment of the invention is cationic/cationogenic precipitation polymers comprising 80-90% by weight of N-vinylpyrrolidone (VP), 0-15% by weight of methacrylamide (MAM), 0.5-3% by weight of ureido methacrylate (UMA), 0-9% by weight of methyl methacrylate (MMA), 4-8% by weight of a cationic/cationogenic monomer c), in particular N-vinylimidazole (VI), 0 to 3% by weight of polyether (meth)acrylate and 0-2% by weight of a crosslinker in copolymerized form, the total amount of all of the copolymerized monomers being 100% by weight, such as, for example:

| Precipitation polymer | VP | MAM | Plex ® 6844-O | VI | Plex ® 6877-O |
|---|---|---|---|---|---|
| K4 | 80 | 10 | 6 | 4 | — |
| K5 | 88 | — | 4 | 6 | 2 |

A further embodiment of the invention is cationic/cationogenic precipitation polymers comprising 80-90% by weight of N-vinylpyrrolidone (VP), 0.1-3% by weight of allyl-D-gluconamide (AGA), 10-18% by weight of a cationic/cationogenic monomer c), in particular N-vinylimidazole (VI), 0-6% by weight of methyl methacrylate, 0.5 to 3% by weight of polyether (meth)acrylate and 0-2% by weight of a crosslinker in copolymerized form, the total amount of all of the copolymerized monomers being 100% by weight, such as, for example:

| Precipitation polymer | VP | AGA | VI | Plex ® 6877-O |
|---|---|---|---|---|
| K6 | 80 | 1 | 15 | 4 |

A further embodiment of the invention is anionic/anionogenic precipitation polymers comprising 85-97% by weight of N-vinylpyrrolidone (VP), 1-4% by weight of methacrylamide (MAM), 1-6% by weight of acrylic acid (AA) and 0-2% by weight of a crosslinker, preferably pentaerythritol triallyl ether (PETAE), the total amount of all of the copolymerized monomers being 100% by weight, such as, for example,

| Precipitation polymer | VP | MAM | AA |
|---|---|---|---|
| A1 | 95 | 3 | 2 |
| A2 | 93 | 2 | 5 |

A further embodiment of the invention is anionic/anionogenic precipitation polymers comprising 85-95% by weight of N-vinylpyrrolidone (VP), 0.1-2% by weight of ureido methacrylate, 0-6% by weight of methyl methacrylate, 5-10% by weight of acrylic acid (AA), 0-3% by weight of polyether (meth)acrylate, 0-9% by weight of methyl methacrylate and 0-2% by weight of a crosslinker in copolymerized form, the total amount of all of the copolymerized monomers being 100% by weight, such as, for example,

| Precipitation | | | | |
|---|---|---|---|---|
| polymer | VP | Plex ® 6844-O | AA | Plex ® 6877-O |
| A3 | 92 | 2 | 6 | |
| A4 | 86 | 4 | 10 | |
| A5 | 80 | 4 | 10 | 6 |

A further embodiment of the invention is anionic/anionogenic precipitation polymers comprising 85-95% by weight of N-vinylpyrrolidone (VP), 0, 1-2% by weight of allyl-D-gluconamide, 8-12% by weight of acrylic acid (AA) and 0-2% by weight of a crosslinker in copolymerized form, the total amount of all of the copolymerized monomers being 100% by weight, such as, for example,

| Precipitation polymer | VP | AGA | AA |
|---|---|---|---|
| A6 | 90 | 1 | 9 |

The precipitation polymers according to the invention with high contents of copolymerized N-vinylpyrrolidone have particularly advantageous properties. These are, for example, the higher molecular weight, the lower stickiness, the increased thickening effect and the better ability to form pulverulent formulations compared to homopolymers of N-vinylpyrrolidone or polymers with comparable contents of copolymerized N-vinylpyrrolidone which are obtained by other polymerization methods, such as, for example, solution polymerization.

Use of the precipitation polymers according to the invention

The invention further provides cosmetic compositions selected from gel creams, hydroformulations, stick formulations, cosmetic oils and oil gels, mascara, self-tanning products, face care compositions, body care compositions, after sun preparations, hair shaping compositions and hair setting agents, which comprise the precipitation polymers according to the invention.

Further cosmetic compositions according to the invention are skin cosmetic compositions, in particular those for caring for the skin, which comprise the precipitation polymers according to the invention. These are present in particular as W/O or O/W skin creams, day and night creams, eye creams, face creams, antiwrinkle creams, mimic creams, moisturizing creams, bleaching creams, vitamin creams, skin lotions, care lotions and moisturizing lotions.

Furthermore, the precipitation polymers according to the invention are suitable as ingredient for skin cosmetic preparations, such as facial toners, face masks, deodorants and other cosmetic lotions and for use in decorative cosmetics, for example as concealing stick, stage make-up, in mascara and eyeshadows, lipsticks, kohl pencils, eyeliners, make-up, foundations, blushers and powders and eyebrow pencils.

Moreover, the precipitation polymers according to the invention can be used in nose strips for pore cleansing, in antiacne compositions, repellents, shaving compositions, hair removal compositions, intimate care compositions, footcare compositions, and also in baby care.

Further preferred preparations according to the invention are washing, showering and bathing preparations which comprise the precipitation polymers according to the invention.

Within the context of this invention, washing, showering and bathing preparations are understood as meaning soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and shower gels, foam baths, oil baths and scrub preparations, shaving foams, shaving lotions and shaving creams.

The cosmetic preparations according to the invention may be present as aqueous or aqueous-alcoholic solutions, O/W and W/O emulsions, hydrodispersion formulations, solids-stabilized formulations, stick formulations, PIT formulations, in the form of creams, foams, sprays (pump spray or aerosol), gels, gel sprays, lotions, oils, oil gels or mousse and can accordingly be formulated with customary further auxiliaries.

The cosmetic preparations according to the invention preferably comprise at least one precipitation polymer according to the invention, at least one cosmetically acceptable carrier and at least one constituent different therefrom which is selected from cosmetically active ingredients, emulsifiers, surfactants, preservatives, perfume oils, further thickeners, hair polymers, hair and skin conditioners, graft polymers, water-soluble or dispersible silicone-containing polymers, photoprotective agents, bleaching agents, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners.

Haircare compositions preferred according to the invention are selected from pretreatment compositions, hair rinses, hair conditioners, hair balsams, leave-on hair treatments, rinse-off hair treatments, hair tonics, pomades, styling creams, styling lotions, styling gels, end fluids, hot-oil treatments and foam treatments.

The precipitation polymers according to the invention are preferably used as rheology-modifying film formers, hair setting agents and conditioners for producing cosmetic, preferably hair cosmetic, preparations.

The invention thus further provides cosmetic, in particular hair cosmetic, preparations comprising the precipitation polymers according to the invention.

Preferred hair cosmetic compositions are hair cleansing compositions, shampoos, haircare compositions and hair setting agents, including in particular hair setting gels.

The precipitation polymers according to the invention are effective in particular as film-forming and/or conditioning rheology modifiers. They are therefore specifically suitable for hair setting agents as “thickening setting agent” or “setting thickener” and in haircare compositions as “conditioning thickener”.

In principle, when used in multiphase compositions such as, for example, O/W and W/O, the precipitation polymers according to the invention can be used either in the water phase or in the oil phase. In general, heterogeneous-phase liquid/liquid compositions comprise the precipitation polymers according to the invention substantially in the water phase.

The invention further provides hair cosmetic compositions comprising

A) at least one precipitation polymer according to the invention,
B) optionally at least one hair polymer different from A),
C) at least one cosmetically acceptable carrier, and
D) optionally at least one cosmetically acceptable active ingredient and/or auxiliary different from A) and B).

The precipitation polymers according to the invention are advantageously characterized not only by film-forming properties, but also by rheology-modifying properties. They can thus be used in hair cosmetic compositions also as a hair-setting component, meaning that the use of further setting polymers is required only in a reduced amount or may even be superfluous altogether.

The precipitation polymers according to the invention are also advantageously characterized by conditioning properties and can improve the sensory properties of the hair, e.g. give it suppleness and shine.

The hair cosmetic compositions comprise the precipitation polymers according to the invention preferably in a fraction of from about 0.1 to 10% by weight, particularly preferably 0.2 to 6% by weight, in particular 0.3 to 3% by weight, based on the total weight of the composition.

Examples of suitable hair polymers B) and preferred amounts thereof are described in detail in WO 2007/010035, p. 68, I.32 to p. 70, I.22. Reference is hereby made to this passage in its entirety.

The compositions preferably have a carrier component C) which is selected from water, hydrophilic components, hydrophobic components and mixtures thereof.

Suitable carrier components C) are described in detail in WO 2007/010035, p. 70, I.28 to p. 71, I.37. Reference is hereby made to this passage in its entirety.

In addition, the compositions according to the invention can comprise, as component D), at least one further cosmetic active ingredient or auxiliary different from A) and B). Suitable components D) are described in detail in WO 2007/010035, p. 72, I.2 to p. 72, I.13. Reference is hereby made to this passage in its entirety.

The precipitation polymers according to the invention can be used together with known thickeners. Suitable thickeners are described in detail in WO 2007/010035, p. 72, I.15 to p. 72, I.24. Reference is hereby made to this passage in its entirety.

### Conditioners

The conditioners chosen for the cosmetic preparations according to the invention are preferably those conditioners which are described on page 34, line 24 to page 37, line 10 of WO 2006/106140, to which reference is hereby made.

### Thickeners

Thickeners suitable for gels, shampoos and haircare compositions are specified in “Kosmetik und Hygiene von Kopf bis Fuβ [Cosmetics and hygiene from head to toe]”, ed. W. Umbach, 3rd edition, Wiley-VCH, 2004, pp. 235-236, to which reference is made at this point in its entirety.

Suitable further thickeners for the cosmetic preparations according to the invention are also described, for example, on page 37, line 12 to page 38, line 8 of WO 2006/106140, to which reference is hereby made.

### Preservatives

Suitable preservatives for the cosmetic compositions according to the invention are described, for example, on page 38, line 10 to page 39, line 18 of WO 2006/106140, to which reference is hereby made.

### UV Photoprotective Filters

Suitable UV photoprotective filters for the cosmetic compositions according to the invention are described, for example, on 39, line 20 to page 41, line 10 of WO 2006/106140, to which reference is hereby made.

### Antioxidants

Suitable antioxidants for the cosmetic compositions according to the invention are described, for example, on page 41, line 12 to page 42, line 33 of WO 2006/106140, to which reference is hereby made.

### Dispersants

If insoluble active ingredients, for example antidandruff active ingredients or silicone oils, are to be dispersed or kept permanently in suspension in the compositions according to the invention, preference is given to using dispersants and thickeners, such as, for example, magnesium aluminum silicates, bentonites, fatty acyl derivatives, polyvinylpyrrolidone or hydrocolloids, e.g. xanthan gum or carbomers.

The compositions can comprise further additives customary in cosmetics, for example perfume, dyes, refatting agents, complexing agents and sequestrants, pearlizing agents, plant extracts, vitamins, active ingredients, pigments which have a coloring effect, softening, moisturizing and/or humectant substances, or other customary constituents, such as alcohols, polyols, polymers, organic acids for pH adjustment, foam stabilizers, electrolytes, organic solvents or silicone derivatives.

As regards the specified further ingredients known to the person skilled in the art for the compositions, reference may be made to “Kosmetik und Hygiene von Kopf bis Fuβ [Cosmetics and hygiene from head to toe]”, ed. W. Umbach, 3rd edition, Wiley-VCH, 2004, pp. 123-128, to which reference is hereby made.

The compositions according to the invention, such as hairsprays, gels, shampoos and haircare compositions, comprise optionally ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glyceryl oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 cocoa glycerides, PEG-40 hydrogenated castor oil, PEG-200 hydrogentated glyceryl palmate. Ethoxylated glycerol fatty acid esters are used in aqueous cleaning formulations for various purposes. Glycerol fatty acid esters with a degree of ethoxylation of ca. 30-50 serve as solubility promoters for nonpolar substances such as perfume oils. Highly ethoxylated glycerol fatty acid esters are used as thickeners.

### Active Ingredients

Advantageous active ingredients for the cosmetic compositions according to the invention are described, for example, on page 44, line 24 to page 49, line 39 of WO 2006/106140, to which reference is hereby made.

### UV Photoprotective Agents

In one preferred embodiment, the compositions according to the invention comprise UV photoprotective agents for protecting the skin and/or the hair. Suitable UV photoprotective agents are described in detail in WO 2006/106114, p. 24, I.4 to p. 27, I.27, to which reference is hereby made in its entirety.

### Pearlescent Waxes

Suitable pearlescent waxes for the cosmetic compositions according to the invention are described, for example, on page 50, line 1 to line 16 of WO 2006/106140, to which reference is hereby made in its entirety.

### Emulsifiers

In one preferred embodiment of the invention, the cosmetic compositions according to the invention are in the form of emulsions. The preparation of such emulsions takes place by known methods. Suitable emulsifiers for the emulsions according to the invention are described, for example, on page 50, line 18 to page 53, line 4 of WO 2006/106140, to which reference is hereby made in its entirety.

### Perfume Oils

If perfume oils are to be added to the cosmetic compositions according to the invention, then suitable perfume oils are described, for example, on page 53, line 10 to page 54, line 3 of WO 2006/106140, to which reference is hereby made in its entirety.

### Pigments

The cosmetic compositions according to the invention optionally further comprise pigments. Suitable pigments for the compositions according to the invention are described, for example, on page 54, line 5 to page 55, line 19 of WO 2006/106140, to which reference is hereby made in its entirety.

### Nanoparticles

The compositions according to the invention optionally comprise water-insoluble nanoparticles, i.e. particles with a particle size in the range from 1 to 200, preferably from 5 to 100 nm. Preferred nanoparticles are nanoparticles of metal oxides, in particular of zinc oxide and/or titanium dioxide.

### Polymers

In one preferred embodiment, apart from the precipitation polymers according to the invention, the cosmetic compositions according to the invention also comprise further polymers. Suitable further polymers are described, for example, on page 55, line 21 to page 63, line 2 of WO 2006/106140. Reference is hereby made to the content of said passage in its entirety.

The precipitation polymers according to the invention are also suitable as rheology-modifying film formers in hair gels, in particular so-called styling gels. Of particular suitability for this purpose are those precipitation polymers which have an excess of anionic and/or anionogenic groups.

The invention thus provides hair gels and hair setting gels which comprise precipitation polymers according to the invention which have an excess of anionic and/or anionogenic groups, i.e. in which the molar amount of anionic and anionogenic groups is greater than the molar amount of cationic and cationogenic groups.

These are in particular anionic/anionogenic precipitation polymers which comprise, in copolymerized form, 85-97% by weight of N-vinylpyrrolidone (VP), 1-4% by weight of methacrylamide (MAM), 1-6% by weight of acrylic acid (AA) and 0-2% by weight of a crosslinker, preferably pentaerythritol triallyl ether (PETAE), the total amount of all of the copolymerized monomers being 100% by weight.

These are also in particular anionic/anionogenic precipitation polymers which comprise, in copolymerized form, 85-95% by weight of N-vinylpyrrolidone (VP), 0.1-2% by weight of ureido methacrylate, 0-6% by weight of methyl methacrylate, 5-10% by weight of acrylic acid (AA), 0-3% by weight of polyether (meth)acrylate, 0-9% by weight of methyl methacrylate and 0-2% by weight of a crosslinker, the total amount of all of the copolymerized monomers being 100% by weight.

These are also in particular anionic/anionogenic precipitation polymers which comprise, in copolymerized form, 85-95% by weight of N-vinylpyrrolidone (VP), 0.1-2% by weight of allyl-D-gluconamide (AGA), 8-12% by weight of acrylic acid (AA) and 0-2% by weight of a crosslinker, the total amount of all of the copolymerized monomers being 100% by weight.

A preferred embodiment of the invention is hair cosmetic preparations, in particular hair setting agents and hair gels which, besides the rheology-modifying precipitation polymers according to the invention, comprise gel formers customary in cosmetics. Such further customary gel formers are slightly crosslinked polyacrylic acid, for example Carbomer (INCI), cellulose derivates, e.g. hydroxypropylcellulose, hydroxyethylcellulose, cationically modified celluloses, polysaccharides, e.g. xanthum gum, caprylic/capric triglycerides, sodium acrylates copolymer, polyquaternium-32 (and) Paraffinum Liquidum (INCI), sodium acrylates copolymer (and) Paraffinum Liquidum (and) PPG-1 trideceth-6, acrylamidopropyl trimonium chloride/acrylamide copolymer, steareth-10 allyl ether acrylates copolymer, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44.

A preferred embodiment of the invention is hair cosmetic preparations, in particular hair setting agents and hair gels, which comprise at least one rheology-modifying anionic/anionogenic precipitation polymer according to the invention and at least one thickener with the INCI name Carbomer. Preferred thickeners with the name Carbomer are available commercially, for example under the trade name Carbopol®.

A further preferred embodiment of the invention is hair cosmetic preparations, in particular hair setting agents and hair gels, which comprise at least one precipitation polymer according to the invention and at least one anionic associative thickener, such as, for example, so-called HASE thickeners (HASE means “anionic hydrophobically modified alkali-soluble acrylic polymer emulsion”) having the INCI names Acrylates/Steareth-20 Methacrylate Copolymer (for example Aculyne®22), Acrylates/Beheneth-25 Methacrylate Copolymer (for example Aculyne®28)) or Acrylates/Steareth-20 Methacrylate Crosspolymer (for example Aculyne®88).

### Hair Washing Compositions

One preferred embodiment of the invention is hair washing compositions and shampoos comprising the precipitation polymers according to the invention. For this embodiment, on account of their conditioning end rheology-modifying properties, in particular the cationic or cationogenic precipitation polymers according to the invention are suitable, i.e. those polymers according to the invention in which the molar amount of anionic and anionogenic groups is less than the molar amount of cationic and cationogenic groups.

These are, for example, cationic/cationogenic precipitation polymers according to the invention which comprise, in copolymerized form, 88-92% by weight of N-vinylpyrrolidone (VP), 3-6% by weight of methacrylamide (MAM), 4-8% by weight of a cationic/cationogenic monomer c), in particular N-vinylimidazole (VI) and 0-2% by weight of a crosslinker, preferably pentaerythritol triallyl ether (PETAE), the total amount of all of the copolymerized monomers being 100% by weight.

These are also cationic/cationogenic precipitation polymers according to the invention which comprise, in copolymerized form, 80-90% by weight of N-vinylpyrrolidone (VP), 0-15% by weight of methacrylamide (MAM), 0.5-3% by weight of ureido methacrylate (UMA), 0-9% by weight of methyl methacrylate (MMA), 4-8% by weight of a cationic/cationogenic monomer c), in particular N-vinylimidazole (VI), 0 to 3% by weight of polyether (meth)acrylate and 0-2% by weight of a crosslinker, the total amount of all of the copolymerized monomers being 100% by weight.

These are also cationic/cationogenic precipitation polymers according to the invention which comprise, in copolymerized form, 80-90% by weight of N-vinylpyrrolidone (VP), 0.1-3% by weight of allyl-D-gluconamide (AGA), 10-18% by weight of a cationic/cationogenic monomer c), in particular N-vinylimidazole (VI), 0-6% by weight of methyl methacrylate, 0.5 to 3% by weight of polyether (meth)acrylate and 0-2% by weight of a crosslinker, the total amount of all of the copolymerized monomers being 100% by weight.

Additional requirements are in some instances placed on shampoos and hair washing compositions depending on hair quality or scalp problem.

Preferred shampoos and hair washing compositions according to the invention comprise anionic surfactants. Further preferred shampoos and hair washing compositions according to the invention comprise combinations of anionic and ampholytic surfactants. Further preferred shampoos and hair washing compositions according to the invention comprise combinations of anionic and zwitterionic surfactants. Further preferred shampoos according to the invention and cosmetic cleansing compositions comprise combinations of anionic and nonionic surfactants. Suitable surfactants of all types have already been described above under “surfactants”.

Preferred anionic surfactants are alkyl sulfates, alkyl polyglycol ether sulfates and ether carboxylic acid salts having 10 to 18 carbon atoms in the alkyl group and up to 12 glycol ether groups in the molecule and sulfosuccinic acid mono- and dialkyl esters having 8 to 18 carbon atoms in the alkyl group and sulfosuccinic acid monoalkyl polyoxyethyl esters having 8 to 18 carbon atoms in the alkyl group and 1 to 6 oxyethyl groups. Particularly preferred anionic surfactants are the alkali metal or ammonium salts of lauryl ether sulfate with a degree of ethoxylation of from 2 to 4 EO units.

A preferred zwitterionic surfactant is the fatty acid amide derivative known under the INCI name Cocamidopropyl Betaine.

Particularly preferred ampholytic surfactants are N-cocoalkyl aminopropionate, cocoacylaminoethyl aminopropionate and N-lauroylsarcosinate.

Preferred nonionic surfactants have proven to be the alkylene oxide addition products onto saturated linear fatty alcohols and fatty acids having in each case 2 to 30 mol of ethylene oxide per mole of fatty alcohol or fatty acid. Preparations with excellent properties are likewise obtained if they comprise fatty acid esters of ethoxylated glycerol as nonionic surfactants.

### Supply

The preparations according to the invention can, for example, be in the form of preparations that can be sprayed from aerosol containers, squeezy bottles or by a pump, spray or foaming device, but also in the form of a composition which can be applied from standard bottles and containers. Suitable propellants for cosmetic or dermatological preparations according to the invention that can be sprayed from aerosol containers are the customary known readily volatile, liquefied propellants, for example dimethyl ether, hydrocarbons (propane, butane, isobutane), which can be used alone or in a mixture, for example mixtures of dimethyl ether and isobutane or dimethyl ether and butane, with one another. Compressed air, nitrogen, nitrogen dioxide or carbon dioxide or mixtures of these substances can also be used advantageously.

The preparations according to the invention can be prepared in the customary manner by mixing the individual constituents. The pH of the preparations can be adjusted in a known manner by adding acids or bases, preferably by adding buffer mixtures, e.g. based on citric acid/citrate or phosphoric acid/phosphate buffer mixtures. In one embodiment of the invention, the pH is below 10, e.g. in the range from 2-7, in particular in the range from 3-5.

Preferred shampoo formulations comprise

a) 0.05 to 10% by weight of at least one precipitation polymer according to the invention,
b) 25 to 94.95% by weight of water,
c) 5 to 50% by weight of surfactants,
d) 0 to 5% by weight of a conditioner,
e) 0 to 10% by weight of further cosmetic constituents.

In a further embodiment, as a result of using the precipitation polymers according to the invention, it is also possible to prepare surfactant-reduced formulations with less than 10% by weight of surfactant, based on the preparation, in a viscosity that suffices for the preparation. In particular, the rheology-modifying precipitation polymers according to the invention are used for establishing the desired viscosity in such preparations, which comprise at least 0.1% by weight and from 0.1 to 10% by weight, preferably less than 10% by weight, of surfactant.

In the shampoos and cosmetic cleansing compositions, all anionic, neutral, amphoteric or cationic surfactants customarily used in shampoos and cosmetic cleansing compositions can be used. Suitable surfactants have been specified above. Particular preference is given to shampoos and cosmetic cleansing compositions with a surfactant content of more than 10% by weight.

In the shampoo formulations, in order to achieve certain effects, further conditioners can be used. These include, for example, cationic polymers with the INCI name Polyquaternium, in particular copolymers of vinylpyrrolidone/N-vinylimidazolium salts (Luviquat®FC, Luviquat®HM, Luviquat®MS, Luviquat®Care), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinylimidazolium salts (Luviquat®Hold); cationic cellulose derivatives (polyquaternium-4 and -10), acrylamide copolymers (polyquaternium-7).

Advantageous conditioners are, for example, the compounds referred to in accordance with INCI as Polyquaternium (in particular Polyquaternium-1 to Polyquaternium-87). The table below gives a non-exhaustive overview of conditioners that can be used in the shampoos and hair washing compositions according to the invention:

| | | | Example |
|---|---|---|---|
| | | | (trade |
| INCI name | CAS number | Polymer type | name) |
| Poly- | CAS 63451-27-4 | urea, N,N′,bis[3-(dimethylamino)- | Mirapol ® A- |
| quaternium-2 | | propyl] polymer with 1,1′-oxybis(2- | 15 |
| | | chloroethane) | |
| Poly- | CAS 26006-22-4 | acrylamide, β-methacryloxyethyl- | |
| quaternium-5 | | triethylammonium methosulfate | |
| Poly- | CAS 26062-79-3 | N,N-dimethyl-N-2-propenyl-2-propen- | Merquat ® |
| quaternium-6 | | aminium chloride (PolyDADMAC) | 100 |
| Poly- | CAS 26590-05-6 | N,N-dimethyl-N-2-propenyl-2-propen- | Merquat ® S |
| quaternium-7 | | aminium chloride, 2-propenamide | |
| Poly- | CAS 53568-66-4, | quaternary ammonium salt of | Celquat ® |
| quaternium-10 | 55353-19-0, | hydroxyethylcellulose | SC-230M, |
| | 54351-50-7, | | Polymer JR |
| | 68610-92-4, | | 400 |
| | 81859-24-7 | | |
| Poly- | CAS 53633-54-8 | vinylpyrrolidone/dimethylaminoethyl | Gafquat ® |
| quaternium-11 | | methacrylate copolymer/diethyl | 755N |
| | | sulfate reaction product | |
| Poly- | CAS 29297-55-0 | vinylpyrrolidone/vinylimidazolinum | Luviquat ® |
| quaternium-16 | | methochloride copolymer | HM552 |
| Poly- | CAS 90624-75-2 | | Mirapol ® |
| quaternium-17 | | | AD-1 |
| Poly- | CAS 110736-85-1 | quaternized water-soluble polyvinyl | |
| quaternium-19 | | alcohol | |
| Poly- | CAS 110736-86-2 | water-dispersible quaternized | |
| quaternium-20 | | polyvinyl octadecyl ether | |
| Poly- | | polysiloxane polydimethyldimethyl- | Abil ® B |
| quaternium-21 | | ammonium acetate copolymer | 9905 |
| Poly- | CAS 53694-17-0 | dimethyldiallylammonium chloride/- | Merquat ® |
| quaternium-22 | | acrylic acid copolymer | 280 |
| Poly- | CAS 107897-23-5 | polymeric quaternary ammonium salt | Quartisoft ® |
| quaternium-24 | | of hydroxyethylcellulose | LM-200 |
| Poly- | CAS 131954-48-8 | vinylpyrrolidone/methacrylamido- | Gafquat ® |
| quaternium-28 | | propyltrimethylammonium chloride | HS-100 |
| | | copolymer | |
| Poly- | CAS 92091-36-6, | chitosan which has been reacted with | Lexquat ® |
| quaternium-29 | 148880-30-2 | propylene oxide and quaternized with | CH |
| | | epichlorohydrin | |
| Poly- | CAS 136505-02- | polymeric, quaternary ammonium salt | Hypan ® |
| quaternium-31 | 7, 139767-67-7 | which is prepared by reacting | QT 100 |
| | | DMAPA acrylates/acrylic acid/ | |
| | | acrylonitrogens copolymer and | |
| | | diethyl sulfate | |
| Poly- | CAS 35429-19-7 | N,N,N-trimethyl-2-([82-methyl-1-oxo- | |
| quaternium-32 | | 2-propenyl)oxy]ethanaminium | |
| | | chloride, polymer with 2-propenamide | |
| Poly- | CAS 26161-33-1 | | |
| quaternium-37 | | | |
| Poly- | | copolymeric quaternary ammonium | |
| quaternium-44 | | salt of vinylpyrrolidone and | |
| | | quaternized imidazoline | |
| Poly- | | polymeric quaternary ammonium salt | SoftCAT ® |
| quaternium-67 | | of hydroxyethyl cellulose reacted with | |
| | | trimethyl ammonium substituted | |
| | | epoxide and a lauryl dimethyl | |
| | | ammonium substituted epoxide | |
| Poly- | | | Polycare ® |
| quaternium-74 | | | Boost |
| Poly- | | | Luviquat ® |
| quaternium-87 | | | Sensation |

The hair gels according to the invention are provided in containers customary for gels, preferably in tubes or small pots.

### EXAMPLES

The invention is described in more detail by the examples below, but is not limited thereto.

Meaning of the abbreviations/trade names:
Acrylic acid AA
Allyl-D-gluconamide AGA
Methacrylamide MAM
Methyl methacrylate MMA
N-Vinylimidazole VI
N-Vinylpyrrolidone VP
Pentaerythritol triallyl PETAE
ether
Ureido methacrylate UMA
Plex® 6844-O 25% by weight of UMA in MMA
Plex® 6877-O 25% by weight of C₁₆₋₁₈-alkyl (EO)₂₅ methacrylate in MMA
AMP 2-amino-2-methylpropanol

Unless expressly determined otherwise, the quantitative data in “%” are percent by weight data.

### Preparation of the Precipitation Polymers

### Preparation Procedure (A) for Example 1

| | | |
|---|---|---|
| Initial charge: | ethyl acetate | 535 g |
| | cyclohexane | 192 g |
| | tert-butyl peroctoate | 1.2 g |
| | feed 1 | 57.3 g |
| | feed 2 | 7 g |
| Feed 1: | N-vinylpyrrolidone | 235.2 g |
| | methacrylamide | 4.8 g |
| | ethyl acetate | 46.5 g |
| Feed 2: | ethyl acetate | 46.5 g |
| | Wako V65 | 0.2 g |
| Feed 3: | ethyl acetate | 140 g |
| | Wako V65 | 0.75 g |
| | tert-butyl peroctoate | 1.2 g |

The initial charge was heated to ca. 62° C. under a nitrogen atmosphere. Feed 1 was then metered in over the course of 3 hours and feed 2 was metered in over the course of 4 hours. The reaction mixture was then held at ca. 62° C. for a further 2 hours with stirring. Then, feed 3 was added over the course of 30 minutes and the mixture was stirred at 65° C. for a further 2.5 hours. The mixture was then firstly heated to 70° C. and polymerized for a further 3 hours and finally heated to 90° C. and afterpolymerized for a further 4 hours. The reaction mixture was then left to cool to room temperature, and the solid was filtered off and dried for 24 hours in vacuo at 75° C.

The preparation of the polymers according to examples 2, 6, 7 and 12 was carried out in an analogous manner.

### Preparation Procedure (B) for Example 4

| | | |
|---|---|---|
| Initial charge: | ethyl acetate | 702 g |
| | tert-butyl peroctoate | 2 g |
| | feed 1 | 62 g |
| | feed 2 | 7 g |
| Feed 1: | N-vinylpyrrolidone | 237.6 g |
| | methacrylamide | 13.2 g |
| | 1-vinylimidazole | 13.2 g |
| | ethyl acetate | 46.8 g |
| Feed 2: | ethyl acetate | 46.8 g |
| | Wako ® V65 | 0.26 g |
| Feed 3: | ethyl acetate | 140.4 g |
| | Wako ® V65 | 0.79 g |
| | tert-butyl peroctoate | 1.32 g |

The initial charge was heated to ca. 60° C. under a nitrogen atmosphere. Feed 1 was then metered in over the course of 3 hours and feed 2 was metered in over the course of 4 hours. The reaction mixture was then held at ca. 60° C. for a further 2 hours with stirring. Then, feed 3 was added over the course of 30 minutes and stirred at 65° C. for a further 2.5 hours. Then, the mixture was firstly heated to 70° C. and polymerized for a further 3 hours and finally heated to 80-85° C. and afterpolymerized for a further 4 hours. The reaction mixture was then left to cool to room temperature, and the solid was filtered off and dried for 24 hours in vacuo at 75° C.

The preparation of the polymers according to examples 3, 9, 10, 11, 13 and 14 was carried out in an analogous manner.

### Preparation Procedure (C) for Example 5

| | | |
|---|---|---|
| Initial charge: | ethyl acetate | 535 g |
| | cyclohexane | 192 g |
| | tert-butyl peroctoate | 1.2 g |
| | feed 1 | 57.3 g |
| | feed 2 | 7 g |
| Feed 1: | N-vinylpyrrolidone | 216 g |
| | methacrylamide | 7.2 g |
| | vinylimidazole | 16.3 g |
| | pentaerythritol triallyl ether | 0.48 g |
| | ethyl acetate | 46.5 g |
| Feed 2: | ethyl acetate | 46.5 g |
| | Wako V65 | 0.2 g |
| Feed 3: | ethyl acetate | 140 g |
| | Wako V65 | 0.75 g |
| | tert-butyl peroctoate | 1.2 g |
| Feed 4: | methyl chloride (gas) | 9 g |

The initial charge was heated to ca. 62° C. under a nitrogen atmosphere. Feed 1 was then metered in over the course of 3 hours and feed 2 was metered in over the course of 4 hours. The reaction mixture was then held at ca. 62° C. for a further 2 hours with stirring. Then, feed 3 was added over the course of 30 minutes and the mixture was stirred at 65° C. for a further 2.5 hours. Then, the mixture was firstly heated to 70° C. and polymerized for a further 3 hours and finally heated to 90° C. and afterpolymerized for a further 4 hours. The reaction mixture was then left to cool to ca. 50° C. with stirring, feed 4 was introduced over the course of ca. 30 minutes. The polymer was then methylated for in each case 1 hour at 70° C. and at 90° C. Unconsumed methyl chloride was then removed by deaeration. The solid was then filtered off and dried for 24 hours in vacuo at 75° C.

The preparation of the polymer according to example 8 was carried out in an analogous manner.

The quaternizable groups of the polymers of examples 5 and 8 are quaternized to ca. 75 mol %.

### Precipitation Polymers According to the Invention:

| | | | Plex ® | | | | Plex ® | | |
|---|---|---|---|---|---|---|---|---|---|
| No. | VP | MAM | 6844-O | AGA | AA | VI | 6877-O | PETAE | Type |
| 1 | 98 | 2 | — | — | — | — | — | — | N1 |
| 2 | 95 | 5 | — | — | — | — | — | — | N2 |
| 3 | 90 | 3 | — | — | — | 7 | — | — | K1 |
| 4 | 90 | 5 | — | — | — | 5 | — | — | K2 |
| 5 | 90 | 3 | — | — | — | 6.8 | — | 0.2 | K3 |
| 6 | 80 | 10 | 6 | — | — | 4 | — | — | K4 |
| 7 | 88 | — | 4 | — | — | 6 | 2 | — | K5 |
| 8 | 80 | — | — | 1 | — | 15 | 4 | — | K6 |
| 9 | 95 | 3 | — | — | 2 | — | — | — | A1 |
| 10 | 93 | 2 | — | — | 5 | — | — | — | A2 |
| 11 | 92 | — | 2 | — | 6 | — | — | — | A3 |
| 12 | 90 | — | — | 1 | 9 | — | — | — | A6 |
| 13 | 86 | — | 4 | — | 10 | — | — | — | A4 |
| 14 | 80 | — | 4 | — | 10 | — | 6 | — | A5 |

### Application Examples

### Hair Gels

All gels were prepared with 2-3% by weight of setting polymer, 0.2 or 0.5% by weight of Carbopol®980 and adjusted to pH ca. 7 with triethanolamine.

To prepare the standard gels (SG1 to SG3), polyvinylpyrrolidone or vinylpyrrolidone-vinyl acetate copolymers were used as setting polymers.

| | | Carbopol ® | | | | |
|---|---|---|---|---|---|---|
| Gel with | Polymer | 908 | Viscosity | | Stickiness* | Setting** |
| polymer | [% by wt.] | [% by wt.] | [Pas] | Clarity | Grade 1-4 | Grade 1-4 |
| SG1 with | 3 | 0.5 | ~25 | clear | 2-3 | 3-4 |
| Luviskol VA | | | | | | |
| 64 | | | | | | |
| SG2 with | 3 | 0.5 | ~40 | almost | 2 | 2 |
| Luviskol | | | | clear | | |
| K90 | | | | | | |
| SG3 with | 2 | 0.5 | ~33 | almost | 2 | 2-3 |
| Luviskol | | | | clear | | |
| K90 | | | | | | |
| Gel with | 3 | 0.5 | ~30 | almost | 1-2 | 2 |
| Polymer N2 | | | | clear | | |
| Gel with | 3 | 0.5 | ~20 | clear | 1 | 1-2 |
| Polymer K2 | | | | | | |
| Gel with | 3 | 0.5 | ~30 | almost | 1-2 | 2 |
| Polymer A4 | | | | clear | | |
| Gel with | 3 | 0.2 | ~30 | almost | 1 | 2 |
| Polymer K3 | | | | clear | | |
| Gel with | 2 | 0.2 | ~25 | slightly | 1 | 2 |
| Polymer K5 | | | | cloudy | | |
| Gel with | 2 | 0.2 | ~30 | almost | 1-2 | 2 |
| Polymer A5 | | | | clear | | |

| | | | | |
|---|---|---|---|---|
| * | ** | Grade | Stickiness | Setting (sensory) |
| | | 1 | not sticky | very good |
| | | 2 | slightly sticky | good |
| | | 3 | sticky | average |
| | | 4 | very sticky | weak |

Examples of cosmetic preparations which comprise the precipitation polymers according to the invention are shown below.

Hair gels with Carbopol ®
Thickening phase (phase 1):

| | |
|---|---|
| Carbopol ® 940 (powder) | 1 g |
| Water | 149 g |
| Euxyl ® K100 | q.s. |
| with triethanolamine (99%) | adjust to pH 6.5-7.2 |

Setting phase (phase 2):

| | |
|---|---|
| Polymer 1 | 6 g |
| Cremophor ® WO:perfume [4:1 w/w] | 0.3 g |
| Water | dissolve to 50 g with water |

Phases 1 and 2 are homogenized separately with stirring; this gives a clear, thick gel (thickening phase) and a setting phase. The setting phase is then slowly stirred into the thickening phase to give a virtually clear, solid gel.

Analogous hair gels are prepared using the polymers of examples 2, 3, 4, 6, 11 and 13.

Hair gels with PVP and Carbopol ®
Thickening phase (phase 1):

| | |
|---|---|
| Carbopol ® 940 (powder) | 1 g |
| Water | 149 g |
| Euxyl ® K100 | q.s. |
| with triethanolamine (99% strength) | adjust to pH 6.5-7.2 |

Setting phase (phase 2):

| | |
|---|---|
| Polymer 1 | 3 g |
| Polyvinylpyrrolidone PVP K90 | 3 g |
| Cremophor ® WO:perfume [4:1 w/w] | 0.3 g |
| Water | dissolve to 50 g with water |

Phases 1 and 2 are homogenized separately with stirring; this gives a clear, thick gel (thickening phase) and a setting phase. The setting phase is then slowly stirred into the thickening phase to give a virtually clear, solid gel.

Analogous hair gels are prepared with the polymers of examples 2, 3, 4, 6, 11 and 13.

Hair gels with associative thickener
Thickening phase (phase 1):

| | |
|---|---|
| Aculyne ® 22 (30%) | 6 g |
| Water | 144 g |
| Euxyl ® K100 | q.s. |
| with AMP (90% strength) | adjusted to pH 6.5-7.2 |

Setting phase (phase 2):

| | |
|---|---|
| Polymer 1 | 6 g |
| Cremophor ® WO:perfume [4:1 w/w] | 0.3 g |
| Water | dissolve and dilute to 50 g |

Phases 1 and 2 are homogenized separately with stirring; this gives a clear, thick gel (thickening phase) and a setting phase. The setting phase is then slowly stirred into the thickening phase to give a virtually clear, solid gel.

Analogous hair gels are prepared with the polymers of examples 2, 3, 4, 5, 6, 7, 8, 9, 10, 11, 12, 13 and 14.

Hair gels with associative thickener and cationic setting polymer
Thickening phase (phase 1):

| | |
|---|---|
| Aculyne 22 (30% strength) | 6 g |
| Water | 144 g |
| Euxyl ® K100 | q.s. |
| with AMP (90% strength) | adjusted to pH 6.5-7.2 |

Setting phase (phase 2):

| | |
|---|---|
| Polymer 1 | 4 g |
| Luviquat ® Supreme | 5 g |
| (20% strength) | |
| Cremophor WO:perfume [4:1 w/w] | 0.3 g |
| Water | dissolve and dilute to 50 g |

Phases 1 and 2 are homogenized separately with stirring; this gives a clear, thick gel (thickening phase) and a setting phase. The setting phase is then slowly stirred into the thickening phase to give a virtually clear, solid gel.

Analogous hair gels are prepared with the polymers of examples 2, 3, 4, 5, 6, 7, 8, 9, 10, 11, 12, 13 and 14.

Hair gels with associative thickener and anionic setting polymer
Thickening phase (phase 1):

| | |
|---|---|
| Aculyne 22 (30% strength) | 6 g |
| Water | 144 g |
| K100 | q.s. |
| with AMP (90% strength) | adjust to pH 6.5-7.2 |

Setting phase (phase 2):

| | |
|---|---|
| Polymer 1 | 3 g |
| Ultrahold ® Strong (100%) | 3 g |
| Cremophor WO:perfume [4:1 w/w] | 0.3 g |

dilute with water to 50 g, then adjust to pH 7.5 to 8 with AMP.

Phases 1 and 2 are homogenized separately with stirring; this gives a clear, thick gel (thickening phase) and a setting phase. The setting phase is then slowly stirred into the thickening phase to give a virtually clear, solid gel.

Analogous hair gels are prepared with the polymers of examples 2, 9, 10, 11, 12, 13 and 14.

| Foam setting agent | [% by wt.] |
|---|---|
| Polymer 3 (powder) | 1.00 |
| Cremophor ® A 25 (Ceteareth 25/BASF) | 0.2 |
| Comperlan ® KD (Coamide DEA/Henkel) | 0.1 |
| Water | 78.7 |
| Adjust to pH 5.5 to 6.5 with lactic acid (90%) | |
| Dimethyl ether | 10.0 |
| Further additives: perfume, preservative | |

Preparation: weigh in and dissolve with stirring, bottle and add propellant gas.

Analogous foam setting agents are prepared with the polymers of examples 4, 5, 6, 7 and 8.

| Shampoos | | % by wt. |
|---|---|---|
| A) | Texapon ® NSO 28% strength | 50.0 |
| | Comperlan ® KD | 1.0 |
| | Polymer 5 | 1.0 |
| | Water | 19.0 |
| | Perfume oil | q.s. |
| | | 47 |
| B) | Water | 28.0 |
| | Sodium chloride | 1.0 |
| | Preservative | q.s. |

Preparation: weigh in and, with stirring, dissolve phases A and B separately and mix, slowly stir phase B into phase A.

Analogous shampoos are prepared with the polymers of examples 6, 7 and 8.

### Skin Cosmetic Preparations

Standard O/W cream

| | % by wt. | CTFA |
|---|---|---|
| Oil phase: | | |
| Cremophor ® A6 | 3.3 | ceteareth-6 (and) stearyl alcohol |
| Cremophor ® A25 | 3.3 | ceteareth-25 |
| Glycerol monostearate s.e. | 2.5 | glyceryl stearate |
| Paraffin oil | 7.5 | paraffin oil |
| Cetyl alcohol | 2.5 | cetyl alcohol |
| Luvitol ® EHO | 3.2 | cetearyl octanoate |
| Vitamin E acetate | 1.0 | tocopheryl acetate |
| Nip-Nip | 0.1 | methyl and propyl 4- |
| | | hydroxybenzoate (7:3) |
| Water phase: | | |
| Polymer 5 | 1.0 | |
| Water | 74.0 | water |
| 1,2-propylene glycol | 1.5 | propylene glycol |
| Germall II | 0.1 | imidazolidinyl urea |

### Preparation:

Weigh in and, with stirring, homogenize oil phase and water phase separately at a temperature of ca. 80° C.; slowly stir water phase into oil phase; slowly cool to room temperature with stirring.

Analogous O/W creams are prepared with the polymers of examples 6, 7, 8, 11, 13 and 14. In the case of polymers 5, 6, 7 and 8, the water phase is adjusted to pH 5-6 with lactic acid, and in the case of polymers 11, 13 and 14 the pH is adjusted to 6.5 to 7.2 with triethanolamine.

Standard day lotion

| | % | CTFA Name |
|---|---|---|
| Oil phase: | | |
| Cremophor ® A6 | 1.5 | ceteareth-6 (and) stearyl alcohol |
| Cremophor ® A25 | 1.5 | ceteareth-25 |
| Glycerol monostearate s.e. | 5.0 | glyceryl stearate |
| Uvinul ® MS 40 | 0.5 | benzophenone-4 |
| Paraffin oil | 3.5 | paraffin oil |
| Cetyl alcohol | 0.5 | cetyl alcohol |
| Luvitol ® EHO | 10.0 | cetearyl octanoate |
| D-Panthenol 50 P | 3.0 | panthenol and propylene glycol |
| Vitamin E acetate | 1.0 | tocopheryl acetate |
| Tegiloxan ® 100 | 0.3 | dimethicone |
| Nip-Nip | 0.1 | methyl and propyl-4- |
| | | hydroxybenzoate (7:3) |
| Water phase: | | |
| Polymer 5 | 0.5 | |
| Water | 71.0 | water |
| 1,2-Propylene glycol | 1.5 | propylene glycol |
| Germall II | 0.1 | imidazolidinyl urea |

### Preparation:

Weigh in and, with stirring, homogenize oil phase and water phase separately at a temperature of ca. 80° C.; slowly stir water phase into oil phase; slowly cool to room temperature with stirring.

Analogous lotions are prepared with the polymers of examples 6, 7 and 8.

1. A polymer obtainable by precipitation polymerization which comprises, in copolymerized form,
(a) 80 to 99.9% by weight of at least one nonionic water-soluble monomer (a) of the formula I 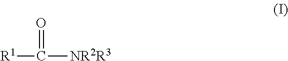
where one of the radicals R¹ to R³ is a group of the formula CH₂═CR⁴— where R⁴═H or C₁-C₄-alkyl and the other radicals R¹ to R³, independently of one another, are H, alkyl, cycloalkyl, heterocycloalkyl, aryl or hetaryl,
where R¹ and R², together with the amide group to which they are bonded, may also be a lactam having 5 to 8 ring atoms,
where R² and R³, together with the nitrogen atom to which they are bonded, may also be a five- to seven-membered heterocycle,
with the proviso that the sum of the carbon atoms of the radicals R¹, R² and R³ is at most 8; and
(b) 0.1 to 20% by weight of a monomer selected from
(b1) methacrylamide,
(b2) radically polymerizable compounds which comprise a structural element of the formula (b2) 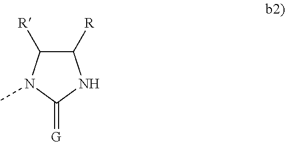
in which R and R′, independently of one another, are hydrogen, alkyl, cycloalkyl, heterocycloalkyl, aryl or hetaryl, where R and R′ may also be covalently bonded, and G is O or NH,
(b3) polyhydroxy compounds carrying at least one allylamino group and
(b4) mixtures thereof,
where the total amount of all of the copolymerized monomers is 100% by weight.

2. The polymer according to claim 1, where the polymer comprises (a) N-vinylpyrrolidone in copolymerized form.

3. The polymer of claim 1, where the polymer comprises from 0.5 to 15% by weight of further monomers (c) different from (a) and (b) in copolymerized form, where the total amount of all of the copolymerized monomers is 100% by weight.

4. The polymer of claim 1, where the polymer comprises N-vinylimidazole in copolymerized form as a monomer (c2).

5. The polymer of claim 1, where the polymer comprises acrylic acid in copolymerized form as a monomer (c1).

6. The polymer of claim 1, where the polymer comprises, in copolymerized form, as monomer (a), N-vinylpyrrolidone and, as monomer (c), (c1) acrylic acid and one or more selected from (c2) N-vinylimidazole, (c3) DMAEMA and (c4) DMAPMAM.

7. The polymer of claim 6, where the weight ratio of (c1) to the sum of (c2), (c3) and (c4) is less than or equal to 1:2 or greater than or equal to 2:1.

8. The polymer of claim 1, where the polymer comprises methacrylamide in copolymerized form as a monomer (b).

9. The polymer of claim 1, where the polymer comprises ureidomethacrylate in copolymerized form as a monomer (b).

10. The polymer of claim 1, where the polymer comprises at least one crosslinker in copolymerized form.

11. A hair cosmetic preparation comprising at least one precipitation polymer of claim 1.

12. A method of treating hair, wherein the hair is brought into contact with a precipitation polymer of claim 1.

13. A method of preparing the polymer of claim 1, wherein the method comprises a precipitation polymerization.

14. A method of preparing the polymer of claim 2, wherein the method comprises a precipitation polymerization.

15. A method of preparing the polymer of claim 6, wherein the method comprises a precipitation polymerization.

16. The polymer of claim 2, where the polymer comprises from 0.5 to 15% by weight of further monomers (c) different from (a) and (b) in copolymerized form, where the total amount of all of the copolymerized monomers is 100% by weight.

17. The polymer of claim 2, where the polymer comprises acrylic acid in copolymerized form as a monomer (c1).

18. The polymer of claim 2, where the polymer comprises at least one crosslinker in copolymerized form.

19. The polymer of claim 6, where the polymer comprises at least one crosslinker in copolymerized form.

20. The polymer of claim 7, where the polymer comprises at least one crosslinker in copolymerized form.
